(19) United States
(12) Patent Application Publication
(10) Pub. No.: US 2024/0099967 A1
(43) Pub. Date: Mar. 28, 2024

(54) INHALABLE COMPOSITION OF CLOFAZIMINE AND METHODS OF USE THEREOF

(71) Applicant: BOARD OF REGENTS, THE UNIVERSITY OF TEXAS SYSTEM, Austin, TX (US)

(72) Inventors: Hugh SMYTH, Austin, TX (US); Ashlee BRUNAUGH, Austin, TX (US)

(21) Appl. No.: 18/458,258

(22) Filed: Aug. 30, 2023

Related U.S. Application Data
(62) Division of application No. 16/652,904, filed on Apr. 1, 2020, now abandoned.
(60) Provisional application No. 62/566,633, filed on Oct. 2, 2017.

(51) Int. Cl. A61K 9/00; A61K 31/498; A61P 31/06

(52) U.S. Cl. CPC A61K 9/0075; A61K 9/0078; A61K 9/008; A61K 31/498; A61P 31/06; A61K 9/48

(57) ABSTRACT

Provided herein is an inhalable composition of clofazimine. Further provided herein are methods of producing the inhalable clofazimine composition by jet milling. Also provided herein are methods of treating pulmonary diseases by administering the inhalable clofazimine composition.

The present application is a divisional of U.S. application Ser. No. 16/652,904, filed Apr. 1, 2020, as a 371 of national phase application under 35 U.S.C. § 371 of International Application No. PCT/US2018/053947, filed Oct. 2, 2018, which claims the priority benefit of U.S. Provisional Application Ser. No. 62/566,633, filed Oct. 2, 2017, the entire contents of each of which is being hereby incorporated by reference.

### BACKGROUND

### 1. Field

The present invention relates generally to the fields of pharmacology and medicine. More particularly, it concerns inhalable clofazimine compositions and methods of their use.

### 2. Description of Related Art

There is a growing and urgent need for new drugs for use against tuberculosis. 10.4 million new TB cases were reported worldwide in 2015, with 580,000 of these cases considered to be multidrug resistance tuberculosis (MDR-TB), defined as M. tuberculosis resistant against rifampicin, or rifampicin and isoniazid (World Health Organization, 2016). In addition, every region of the world has exhibited cases of extensively-drug resistant TB (XDR-TB), defined as M. tuberculosis resistant against isoniazid and rifampicin, plus any fluoroquinolone and at least one of three injectable second-line drugs (amikacin, kanamycin, or capreomycin) (World Health Organization, 2016). With the advent of globalization and mass-migration from high burden areas, these resistant strains are expected to spread. As treatment options dwindle, the reformulation of poorly tolerable, highly active anti-infective agents such as clofazimine (CFZ) is a potential method to overcome resistant TB particularly if they can be targeted to the infection site. Several challenges exist in the development of such formulations. In order to be effectively implemented in the low resource countries in which TB predominates, any potential treatment must be cost-effective as well as easily transported and administered. Additionally, a potential treatment must exhibit a high specificity towards alveolar macrophages through which the M. tuberculosis infection is initiated and propagated (Bloom, 1994). Infectious bacilli are inhaled as droplets and phagocytosed by alveolar macrophages and survive the hostile intracellular environment by restricting acidification of the macrophage and limiting lysosome fusion. In chronic infection, this mechanism leads to a stable population of intracellular mycobacterium (Russel, 2007).

Clofazimine is a weakly basic iminophenazine antibiotic that exhibits activity against mycobacterium, such as Mycobacterium leprae, Mycobacterium avium complex (MAC), and M. tuberculosis with a minimum inhibitory concentration (MIC) ranging from 0.125 to 2 μg/mL (Arbriser et al., 1995, Gangadharam et al., 1992; Lindholm-Levy et al., 1998; Shafran et al., 1996; Kemper et al., 1992; Twomey et al., 1957; Schön et al., 2011; Diacon et al., 2015; Cavanaugh et al., 2017). Importantly, clofazimine exhibits activity against drug-resistant TB and is now recommended as a 2nd-line agent by the World Health Organization in treatment of MDR-TB (World Health Organization, 2016; Cavanaugh et al., 2017; Rastogi et al., 1996; Reddy et al, 1996). Clofazimine may also be used for the treatment of Methicillin-resistant Staphylococcus aureus (MRSA) and inflammatory lung disorders. Clofazimine also exhibits numerous other properties that may be highly beneficial in the treatment of TB, including shorter duration of therapy, synergy with other antimicrobial agents such as pyrazinamide, rifampin, fluoroquinolones, and amikacin that results in enhanced bactericidal activity against stationary phase bacilli, and anti-inflammatory activity (Tyagi et al., 2015; Zhang et al., 2017; Cholo et al., 2017). In particular, clofazimine demonstrates a unique affinity for macrophage uptake and sequestration. Upon uptake of the drug, macrophages transform clofazimine into liquid crystal structures bounded by a bilayer membrane (Baik and Rosania, 2012; Baik et al., 2013). These unique intracellular clofazimine structures may serve as a protective mechanism against cytotoxicity and allow for the mobilization and accumulation of drug at the site of infection in order to maximize therapeutic efficacy (Baik and Rosania, 2012; Baik et al., 2013; Yoon et al., 2016; Yoon et al., 2015).

Though highly active against mycobacterium, the therapeutic efficacy of the existing commercial clofazimine oral formulation (Lamprene®, Novartis) is limited by its poor water solubility (10 mg/L), slow onset of action, and significant adverse effect profile. Oral bioavailability ranges from 45-62%, and exhibits a high degree of inter-patient variability and food effect (Bolla and Nangia, 2012; Clofazimine, 2008; Nix et al., 2004; Holdiness, 1989). Additionally, clofazimine exhibits pH dependent solubility, with pKa values of 2.31 and 9.29 (Keswani et al., 2015). The change in pH that accompanies the transition from the stomach to intestinal environment can potentially lead to recrystallization and precipitation of clofazimine and reduce systemic absorption. At least 30 days of administration is necessary to reach steady-state concentrations, necessitating the use of large loading doses, and a delay in bactericidal activity occurs for up to two weeks after oral dosing, regardless of the dose administered (Holdiness, 1989; Swanson et al., 2015). The necessary high systemic doses are associated with adverse effects that include reddish-brown skin and conjunctiva discoloration (75-100%), GI distress (40-50%) including abdominal pain, nausea, diarrhea, vomiting, and severe complications such as splenic infarction, bowel obstruction, and fatal bleeding secondary to accumulation of crystalline deposits (Novartis, 2006). Additionally, availability of the oral formulation is limited. In the US, Lamprene® is only available for treatment of MDR-TB through single-patient Investigation New Drug applications (INDs) administered by the US Food and Drug Administration (FDA) (Cunningham, 2004; Clofazimine, 2009). Clearly, there is a need to reduce the undesirable systemic adverse effects and improve therapeutic efficacy, as well as a need for a more targeted formulation of clofazimine.

### SUMMARY

In a first embodiment, the present disclosure provides a pharmaceutical composition comprising micronized clofazimine particles with a median particle diameter of 0.5 to 10 μm, wherein the composition comprises less than 10% amorphous material. In some aspects, the composition is a dry powder. In specific aspects, the dry powder is formulated for inhalation. In specific aspects, the composition comprises a single active ingredient, wherein the single active agent is clofazimine.

In particular aspects, the composition is substantially free of excipients. In some aspects, the composition is essentially free of excipients. In particular aspects, the composition is free of added excipients. In specific aspects, the composition is free of excipients. In some aspects, the composition is free of excipients, additives, diluents, carriers, and adjuvants. In specific aspects, the composition is free of one or more of sugars, lubricants, antistatic agents, anti-adherents, glidants, amino acids, peptides, surfactants, lipids (e.g., leucine, isoleucine, lysine, valine, and/or methionine), and phospholipids. In particular aspects the composition is free or essentially free of DMSO, cyclodextrin, dipalmitoylphosphatidylcholine (DPPC), lactose, magnesium stearate, and colloidal silica. The composition may be free or essentially free of DMSO, cyclodextrin, dipalmitoylphosphatidylcholine (DPPC), magnesium stearate, and colloidal silica. The composition may comprise lactose, such as at a concentration of up to 10% by weight, such as 0.1-10% per weight, such as 0.5, 1, 2, 3, 4, 5, 6, 7, 8, 9 or 10% by weight.

In some aspects, the micronized clofazimine particles are substantially crystalline. In particular aspects, the micronized clofazimine particles are essentially crystalline. In certain aspects, the micronized clofazimine particles are crystalline.

In specific aspects, the composition comprises at least 90%, 91%, 92%, 93%, 94%, or 95%, such as 96%, 97%, 98%, 99%, or 100%, by weight of the micronized clofazimine particles.

In particular aspects, the micronized clofazimine particles comprise a median particle diameter of 0.5 to 5 um, such as 0.75 to 4 um, particularly 1 to 3 um. In some aspect, at least 80% of the micronized clofazimine particles comprise a volume equivalent diameter of 1 to 3 um. In some aspects, the micronized clofazimine particles form aggregates. The composition may have a specific surface area of 1.9 to 2.3 m²/g, such as 2.1-2.2 m²/g, such as 2.11, 2.12, 2.13, 2.14, 2.15, 2.16, 2.17, 2.18, 2.19, or 2.2 m²/g. The composition may have a compressibility index of 32 to 37, particularly 33.9-34.0, such as 33.91, 33.92, 33.93, 33.94, 33.94, 33.95, 33.96, 33.97, 33.98, 33.99, or 34.0. The composition may have a Hausner ratio of 10-20, such as 11, 12, 13, 14, 15, 16, 17, 18, 19, or 20. The composition may have an angle of response of 15° to 30°, particularly, 21-23°, such as 22.1, 22.2, 22.3, 22.4, 22.5, 22.6, 22.7, 22.8, 22.9, or 23.0°.

In some aspects, the composition comprises a fine particle fraction (FPF) of at least 50%, such as at least 55%, 60%, 65%, 70%, 75%, or 80%. In certain aspects, the composition comprises a dissolution rate of less than 30% in 24 hours in phosphate buffered saline pH 7.4 with 0.2% polysorbate 80 dissolution medium. In some aspects, the composition is not encapsulated in liposomes.

In certain aspects, the composition comprises less than 5% amorphous material. In particular aspects, the composition is substantially free of amorphous material. In some aspects, the composition is essentially free of amorphous particles as determined by x-ray diffraction or differential scanning calorimetry.

In some aspects, the composition is produced by jet milling, such as air jet milling. In particular aspects, the composition is not produced by spray-drying or ultrasonic homogenization.

In further aspects, the composition is packaged as a unit dosage form. For example, the unit dosage form may be packages as a cartridge, blister, or capsule. In particular aspects, the unit package dose is free of excipients. In some aspects, the unit dosage form comprises 5-30 mg (e.g., 6, 7, 8, 9, 10, 12, 13, 14, 15, 16, 17, 18, 19, 20, 21, 22, 23, 24, 25, 26, 27, 28, 29, or 30 mg) of micronized clofazimine particles. In some aspects, the unit dosage form comprises at least 10 mg of micronized clofazimine particles. In particular aspects, the unit dosage form comprises at least 20 mg of micronized clofazimine particles.

In additional aspects, the dry powder is loaded in a dry powder inhaler such as a simple dry powder inhaler. In some aspects, the dry powder inhaler is an active inhaler. In other aspects, the dry powder inhaler is a passive inhaler. In some aspects, the simple dry powder inhaler comprises less than 10 parts. In one specific aspect, the simple dry powder inhaler is a RSO1 monodose dry powder inhaler. In some aspects, the dry powder inhaler comprises an air flow resistance of 0.01 kPa^{0.5} min/L and 0.06 kPa^{0.5} min/L, such as 0.02 kPa^{0.5} min/L and 0.04 kPa^{0.5} min/L.

Further provided herein is a powder for use in a dry powder inhaler, the powder comprising the micronized clofazimine particle composition of the embodiments, such as an excipient-free inhalable clofazimine composition.

In another embodiment, there is provided a composition comprising a unit dosage form of micronized clofazimine particles, wherein the particles comprise a median particle diameter of 0.5 to 10 μm and the composition is substantially free of excipients. In some aspects, the unit dosage form comprises a composition of micronized clofazimine particles of the embodiments. In some aspects, the unit dosage form is comprised in a cartridge, blister, or capsule. In certain aspects, the unit dosage form comprises at least 10 mg of micronized clofazimine particles. In particular aspects, the unit dosage form comprises at least 20 mg of micronized clofazimine particles.

In yet another embodiment, there is provided a dry powder inhaler comprising a unit dosage form of the embodiments. In some aspects, the dry powder inhaler is a simple dry powder inhaler. In particular aspects, the simple dry powder inhaler comprises less than 10 parts. For example, the simple dry powder inhaler is a RSO1 monodose dry powder inhaler. In specific aspects, the dry powder inhaler comprises an air flow resistance of 0.02 kPa^{0.5} min/L and 0.04 kPa^{0.5} min/L. In some aspects, the dry powder inhaler delivers an emitted dose of 10 to 20 mg with one actuation of the device. In particular aspects, the dry powder inhaler delivers a fine particle dose of 5 to 15 mg with one actuation of the device. In some aspects, the fine particle dose is at least 50%, such as at least 60% or 70%, of the emitted dose with one actuation of the device. In particular aspects, a change in pressure drop across the device from kPa to 1 kPa does not result in a decrease in emitted dose by more than 25%. In specific aspects, a change in pressure drop across the device from 4 kPa to 1 kPa does not result in a decrease in fine particle dose by more than 15%.

In a further embodiment, there is provided a method of preparing the composition of the embodiments (e.g., a composition comprising micronized clofazimine particles), comprising obtaining clofazimine; subjecting the clofazimine to a jet mill; and collecting micronized clofazimine particles with a median particle diameter of 0.5 to 10 μm, wherein the method does not comprise the addition of an excipient. In some aspects, the jet mill is further defined as an air jet mill. In particular aspects, the method does not comprise the addition of a solvent. In additional aspects, the method further comprises loading the micronized clofazimine particles into a dry powder inhaler. In particular aspects, the dry powder inhaler is a simple dry powder inhaler.

Another embodiment provides a method for treating or preventing a pulmonary infection in a patient comprising administering an effective amount of the micronized clofazimine particles composition of the embodiments to the patient.

In some aspects, administering comprises inhaling the micronized clofazimine particles into the patient's lungs. In certain aspects, inhaling comprises the use of an inhaler. In some aspects, the inhaler is a dry powder inhaler, metered dose inhaler, or a nebulizer.

In certain aspects, the pulmonary infection is a bacterial infection. In particular aspects, the pulmonary infection is a mycobacterial infection. In some aspects, the mycobacterial infection is a Mycobacterium tuberculosis infection, Mycobacterium abscesses infection, Mycobacterium kansasii infection or a Mycobacterium avium complex infection. In particular aspects, the Mycobacterium tuberculosis is multidrug resistant. In some aspects, the Mycobacterium tuberculosis is extensively drug resistant. In some aspects, the pulmonary infection is a latent infection. In particular aspects, the Mycobacterium tuberculosis infection is latent. In some aspects, the pulmonary infection is pneumonia, such as methicillin resistant Staphylococcus aureus-associated, or a cystic fibrosis-associated infection.

In additional aspects, the method further comprises administering at least a second therapeutic agent. In some aspects, the at least a second agent is selected from the group consisting of bedaquilline, pyrazinamide, a nucleic acid inhibitor, a protein synthesis inhibitor, and a cell envelope inhibitor. In certain aspects, the protein synthesis inhibitor is linezolid, clarithromycin, amikacin, kanamycin, capreomycin, or streptomycin. In some aspects, the cell envelope inhibitor is ethambutol, ethionamide, thioacetizone, isoniazid, imipenem, clavulanate, cycloserine, terizidone, amoxicillin, or prothionamide. In some aspects, the nucleic acid inhibitor is rifampicin, rifabutin, rifapentine, 4-aminosalicylic acid, moxifloxacin, ofloxacin, or levofloxacin. The second therapeutic agent may be administered separately from the clofazimine particle composition, such as via the rectal, nasal, buccal, vaginal, subcutaneous, intracutaneous, intravenous, intraperitoneal, intramuscular, intraarticular, intrasynovial, intrasternal, intrathecal, intralesional, or intracranial route, or via an implanted reservoir. The second therapeutic agent may be administered prior to or after the clofazimine particle composition.

In particular aspects, the micronized clofazimine particles composition is administered more than once, such as once a day, every other day, every 3 days, or weekly.

In another embodiments, there is provided a method for treating cancer in a patient comprising administering an effective amount of the micronized clofazimine particles composition of the embodiments to the patient. In some aspects, the cancer is lung cancer.

In additional aspects, the method further comprises administering an anti-cancer agent. In some aspects, the anti-cancer agent is chemotherapy, radiotherapy, gene therapy, surgery, hormonal therapy, anti-angiogenic therapy or cytokine therapy.

In certain aspects, administering comprises inhaling the micronized clofazimine particles into the patient's lungs. In particular aspects, inhaling comprises the use of an inhaler. In some aspects, the inhaler is a dry powder inhaler, a metered dose inhaler, or a nebulizer. In particular aspects, the micronized clofazimine particles composition is administered more than once.

In yet another embodiment, there is provided a method for reducing lung inflammation in a patient comprising administering an effective amount of the micronized clofazimine particles composition of the embodiments to the patient. In some aspects, the lung inflammation is associated with asthma, COPD, idiopathic pulmonary fibrosis, or cystic fibrosis. In particular aspects, administering comprises inhaling the micronized clofazimine particles into the patient's lungs. In some aspects, inhaling comprises the use of an inhaler. In some aspects, the inhaler is a dry powder inhaler, metered dose inhaler, or nebulizer. In particular aspects, the micronized clofazimine particles composition is administered more than once.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: SEM images of excipient-free clofazimine spray dried in organic solvents.

FIG. 2: X-ray crystallography diffraction data for clofazimine spray dried in organic solvents.

FIG. 3: Schematic of the Aljet mill used for the micronization of clofazimine.

FIG. 4: Particle size distributions collected from different areas of the Aljet jet mill.

FIGS. 5A-5E: Scanning electron microscopy images of clofazimine crystals. A) unprocessed clofazimine; B) micronized clofazimine particles collected from the collection vessel region of the Aljet mill; C) micronized clofazimine particles collected from the collection vessel region of the Aljet mill dispersed using application of 3 bar air pressure from a Sympatec RODOS disperser unit; D) micronized clofazimine particles collected from the cyclone region of the Aljet mill; E) micronized clofazimine particles collected from the cyclone region of the Aljet mill dispersed using application of 3 bar air pressure from a Sympatec RODOS disperser unit.

FIG. 6: X-ray crystallography diffraction and differential scanning calorimetry data for milled and unprocessed clofazimine.

FIGS. 7A-7C: Particle fractions recovered as a fraction of recovered mass and Mass Median Aerodynamic Diameter (MMAD) determination. A) Emitted fraction (EF %), fine particle fraction less than 5 μm aerodynamic diameter (FPF<5 μm), fraction less than 3 μm aerodynamic diameter (FPF<3 μm), and MMAD for milled clofazimine particles with a geometric volume median diameter of 2.69 μm and 1.81 μm; B) EF %, FPF<5 μm, FPF<3 μm, and MMAD for milled clofazimine particles with a geometric volume median diameter of 1.81 μm aerosolized under conditions of a 4 kPa pressure drop through a low resistance RS01 DPI and 1 kPa pressure drop through a low resistance RS01 DPI; C) Next Generation Impactor (NGI) stage deposition patterns for milled clofazimine.

FIG. 8: Angle of repose analysis of excipient-free milled clofazimine.

FIG. 9: Macrophage phagocytosis of milled clofazimine occurs at a logarithmic rate.

FIG. 10: J774.A1 macrophages exposed to milled clofazimine for 24 hours exhibited a significant population of cells fluorescent at 660 nm emission, which is indicative of intracellular biotransformation of clofazimine.

FIG. 11: Cell proliferation relative to control following treatment with indicated amount of milled or unmilled clofazimine.

FIG. 12: Dissolution of milled clofazimine.

### DESCRIPTION OF ILLUSTRATIVE EMBODIMENTS

Since the adverse effects of CFZ are dose-related and more frequently GI related, administration of CFZ by an alternative route may alleviate or at least limit its side effects. In particular, delivery of CFZ via the inhalation route would be highly beneficial, given that initiation and propagation of TB and NTM infections occurs within the intracellular environment of alveolar lung macrophages. In contrast to oral dosing, direct targeting of CFZ to the lungs via inhalation could be used to rapidly achieve therapeutic drug concentrations at the infection site by taking advantage of the natural clearance mechanism of the lung, alveolar macrophage phagocytosis, to target drug particles to intracellular bacterium. The utilization of a dry powder inhaler for delivery of CFZ is especially favorable, as the product does not require a cold chain supply and is thus well suited for administration in resource-poor regions.

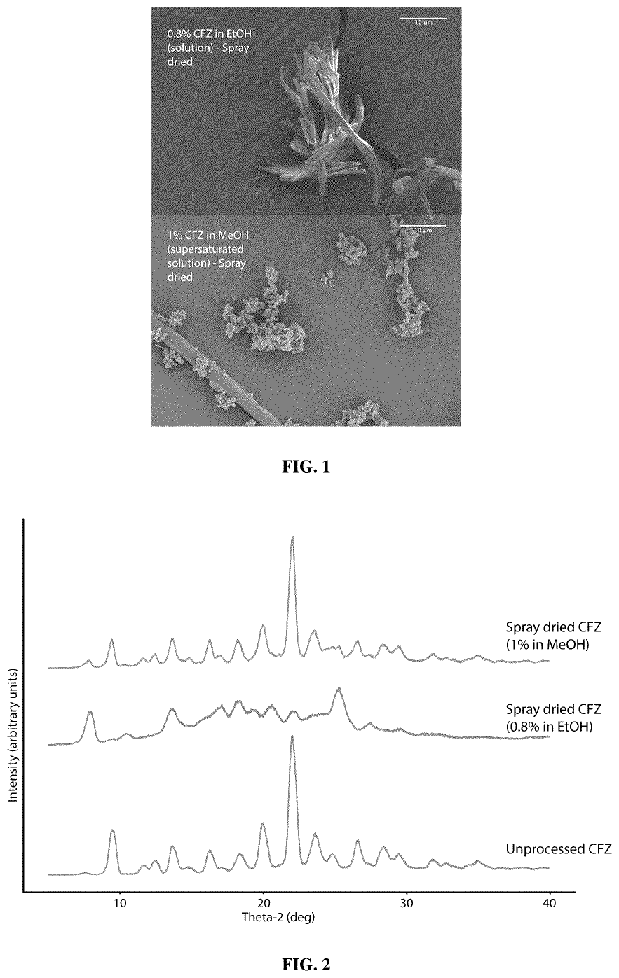

Solubility is a major limiting factor to the development of a pharmaceutically acceptable formulation of CFZ. CFZ is practically insoluble in water. Additionally, this highly beneficial antibiotic exhibits limited solubility in a variety of other solvents. According to the Merck Index, clofazimine is soluble in DMF and benzene, soluble in 15 parts chloroform, 700 parts ethanol, 1000 parts ether, sparingly soluble in acetone and ethyl acetate and practically insoluble in water. It has also been reported that a 0.1% clofazimine solution in methanol can be formed (Sabnis et al., 2015). The International Council for Harmonization of Technical Requirements for Pharmaceuticals for Human Use (ICH) guidance for industry Q3C Impurities: Residual Solvents recognizes benzene as a Class 1 solvent (should not be employed in the manufacture of drug products; 2 ppm concentration limit), chloroform, methanol, acetonitrile and are Class 2 solvents (should be limited in drug products due to inherent toxicity; 60 ppm, 3000 ppm, and 410 ppm, respectively), and dilute acetic acid and ethanol are listed as recognized as Class 3 solvents. Considering both the large volumes required for full dissolution as well as the safety limitations of the use of these solvents, manufacturing of respirable CFZ particles via commonly used constructive (bottom-up) particle engineering techniques for dry powder formulation such as spray drying is extremely challenging. Successful spray drying of respirable CFZ particles is reported to require addition of excipients to the formulation, such as leucine or dipalmitoylphosphatidylcholine (DPPC), in order to formulate a product suitable for lung deposition (Germishuizen et al. 2013; Sabnis, 2015). Spray drying of pure CFZ in organic solvents such as ethanol or methanol results in formation of poorly dispersible needle-shaped crystals (FIG. 1). If a supersaturated solution of CFZ is formulated for the liquid feed, a multimodel size distribution results, potentially due to the drug precipitating out of the liquid feed prior to complete drying of the droplets. If a saturated solution is formulated for the organic solvent feed, defined as complete dissolution of CFZ in the solvent, a partially amorphous formulation of CFZ results, which is prone to physicochemical instability (FIG. 2). Thus, methods to overcome these limitations are needed.

Accordingly, in some embodiments, the present disclosure provides an excipient-free clofazimine dry powder composition for inhalation. The present inhalable clofazimine composition may have particles within a median particle diameter range of 0.5-10 μm, particularly in the range of 0.75-4 μm which allows for efficient aerosolization for lung delivery. In particular, the present composition can provide high doses regardless of patient inhalation flow rates, such as from a simple passive dry powder inhalation device. In addition, the clofazimine particles can be rapidly and efficiently uptaken into alveolar macrophages which allows for targeting of intracellular infections and providing a drug reservoir for sustained release and anti-infective activity. The present studies found that the micronized clofazimine is rapidly transformed into a low toxicity and anti-inflammatory crystalline-like form when taken up by alveolar macrophages. This crystalline-like form is beneficial for rapid onset of action of therapeutic effects, which can be delayed for up to two weeks in currently available dosage forms.

Further, the low aqueous solubility of the present composition limits lung dissolution and systemic absorption, thereby reducing systemic side effects. Upon delivery to the macrophages, crystals undergo biotransformation and sequestration results, which is associated with anti-inflammatory activity and accumulation at the site of action. As compared to the solubilized form of the drug, the present composition has reduced macrophage toxicity. In addition, the present composition is substantially free of amorphous particles which can result from methods such as spray drying and lead to too rapid dissolution and drug precipitation. Indeed, the present composition decreases solubility and allows for macrophage uptake of particles.

The present disclosure further provides methods for producing the inhalable clofazimine composition by subjecting commercially available raw clofazimine crystals to jet milling, such as air jet milling, and collecting fractions of clofazimine within a specific median particle diameter range, such as 0.5-10 μm, particularly less than 5 μm. In some aspects, the output clofazimine may be re-applied to the mill for increasing the fine particle fraction. Thus, the present method is a mechanically simple, environmentally-friendly, and cost-effective micronization method for the producing the clofazimine dry powder composition.

Further embodiments provide methods of treating or preventing diseases by administering the inhalable clofazimine composition provided herein. For example, the therapy may be used to treat pulmonary infections, such as TB lung infections including latent infections, pneumonia (e.g., MRSA), cystic fibrosis lung infections, inflammatory lung infections, and lung cancer. In particular, inhalable clofazimine may be used to treat mycobacterium infections.

### II. Definitions

A used herein, the term “substantially free,” means that a composition contains less than 1% of a component (e.g., excipient) other than the active agent (e.g., clofazimine).

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is preferably below 0.01%. Most preferred is a composition in which no amount of the specified component can be detected with standard analytical methods.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

The terms “about”, “substantially” and “approximately” mean, in general, the stated value plus or minus 5%.

As used herein in the specification and the claims, the term “micronize” or “micronized” is used to indicate that a substance is to be, or has been, broken down into very fine particles, typically less than 10 μm, preferably between 0.5 and 5 urn, more preferably between 1 and 3 urn. A substance may be micronized by milling, grinding, or crushing. Milling may be performed by any method known in the art, such as by air jet mill, ball mill, wet mill, high pressure homogenization, or cryogenic mill.

As used herein in the specification and the claims, the term “air jet mill” refers to a device or method for reducing particle size by using a jet of compressed gas to impact particles into one another or the walls of the mill, thereby pulverizing the particles. An air jet mill may be used to micronize particles. Air jet mills are commercially available, such as the Aljet Model 00 Jet-O-Mizer™ (Fluid Energy, Telford, PA).

As used herein in the specification and the claims, the term “ball mill” refers to a device or method for reducing particle size by adding the particle of interest and a grinding medium to the interior of a cylinder and rotating the cylinder. The particles of interest are broken down as the grinding medium rises and falls along the exterior of the cylinder as it rotates.

As used herein in the specification and the claims, the term “wet mill” or “media mill” refers to a device or method for reducing particle size by adding the particle of interest to device with an agitator, containing a media comprising a liquid and a grinding medium. With the addition of the particle of interest, as the agitator rotates, the energy it disperses causes the grinding medium and particles of interest to come into contact and break down the particles of interest.

As used herein in the specification and the claims, the term “high pressure homogenization” refers to a method of reducing particle size by adding the particle of interest to a device which combines both pressure and mechanical forces to break down the particle of interest. Mechanical forces used in high pressure homogenization may include impact, shear, and cavitation, among others.

As used herein in the specification and the claims, the term “cryogenic mill” refers to a device or method for reducing particle size by first chilling a particle of interest with dry ice, liquid nitrogen, or other cryogenic liquid, and subsequently milling the particle of interest to reduce the size.

The terms “compositions,” “pharmaceutical compositions,” “formulations,” and “preparations” are used synonymously and interchangeably herein.

The term “clofazimine” refers to N,5-bis(4-chlorophenyl)-3-(1-methylethylimino)-5H-phenazin-2-amine in any of its forms, including non-salt and salt forms (e.g., clofazimine mesylate), esters, anhydrous and hydrate forms of non-salt and salt forms, solvates of non-salt and salts forms, its enantiomers (R and S forms, which may also by identified as d and 1 forms), and mixtures of these enantiomers (e.g., racemic mixture, or mixtures enriched in one of the enantiomers relative to the other).

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

“Subject” and “patient” refer to either a human or non-human, such as primates, mammals, and vertebrates. In particular embodiments, the subject is a human.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

The term “derivative thereof” refers to any chemically modified polysaccharide, wherein at least one of the monomeric saccharide units is modified by substitution of atoms or molecular groups or bonds. In one embodiment, a derivative thereof is a salt thereof. Salts are, for example, salts with suitable mineral acids, such as hydrohalic acids, sulfuric acid or phosphoric acid, for example hydrochlorides, hydrobromides, sulfates, hydrogen sulfates or phosphates, salts with suitable carboxylic acids, such as optionally hydroxylated lower alkanoic acids, for example acetic acid, glycolic acid, propionic acid, lactic acid or pivalic acid, optionally hydroxylated and/or oxo-substituted lower alkanedicarboxylic acids, for example oxalic acid, succinic acid, fumaric acid, maleic acid, tartaric acid, citric acid, pyruvic acid, malic acid, ascorbic acid, and also with aromatic, heteroaromatic or araliphatic carboxylic acids, such as benzoic acid, nicotinic acid or mandelic acid, and salts with suitable aliphatic or aromatic sulfonic acids or N-substituted sulfamic acids, for example methanesulfonates, benzenesulfonates, p-toluenesulfonates or N-cyclohexylsulfamates (cyclamates).

The term “dissolution” as used herein refers to a process by which a solid substance, here the active ingredients, is dispersed in molecular form in a medium. The dissolution rate of the active ingredients of the pharmaceutical dose of the invention is defined by the amount of drug substance that goes in solution per unit time under standardized conditions of liquid/solid interface, temperature and solvent composition.

An “active ingredient” (AI) (also referred to as an active compound, active substance, active agent, pharmaceutical agent, agent, biologically active molecule, or a therapeutic compound) is the ingredient in a pharmaceutical drug that is biologically active. The similar terms active pharmaceutical ingredient (API) and bulk active are also used in medicine.

As used herein, “excipient” refers to pharmaceutically acceptable carriers that are relatively inert substances used to facilitate administration or delivery of an API into a subject or used to facilitate processing of an API into drug formulations that can be used pharmaceutically for delivery to the site of action in a subject. Non-limiting examples of excipients include stabilizing agents, surfactants, surface modifiers, solubility enhancers, buffers, encapsulating agents, antioxidants, preservatives, nonionic wetting or clarifying agents, viscosity increasing agents, and absorption-enhancing agents.

As used herein, the term “aerosols” refers to dispersions in air of solid or liquid particles, of fine enough particle size and consequent low settling velocities to have relative airborne stability (See Knight, V., Viral and Mycoplasmal Infections of the Respiratory Tract. 1973, Lea and Febiger, Phila. Pa., pp. 2). “clofazimine aerosols” consist of micronized clofazimine, which is essentially excipient free, intended for delivery into the respiratory tract of a person or animal.

As used herein, “inhalation” or “pulmonary inhalation” is used to refer to administration of pharmaceutical preparations by inhalation so that they reach the lungs and in particular embodiments the alveolar regions of the lung. Typically inhalation is through the mouth, but in alternative embodiments in can entail inhalation through the nose.

As used herein, “dry powder” refers to a fine particulate composition that is not suspended or dissolved in an aqueous liquid.

A “simple dry powder inhaler” refers a device for the delivery of medication to the respiratory tract, in which the medication is delivered as a dry powder in a single-use, single-dose manner. In particular aspects, a simple dry powder inhaler has fewer than 10 working parts. In some aspects, the simple dry powder inhaler is a passive inhaler such that the dispersion energy is provided by the patient's inhalation force rather than through the application of an external energy source.

A “median particle diameter” refers to the geometric diameter as measured by laser diffraction or image analysis. In some aspects, at least 80% of the particles by volume are in the median particle diameter range.

A “Mass Median Aerodynamic Diameter (MMAD)” refers to the aerodynamic diameter (different than the geometric diameter), and is measured by cascade impaction or time of flight.

The term “amorphous” refers to a noncrystalline solid wherein the molecules are not organized in a definite lattice pattern. In some aspects, fewer than 10% of the composition may be an amorphous solid form of clofazimine.

### III. Clofazimine Composition for Inhalation

In particular embodiments, the present disclosure provides an inhalable clofazimine (or a derivative or pharmaceutically acceptable salt thereof) composition. The clofazimine composition may be produced by jet milling of native clofazimine to produce crystalline clofazimine particles for inhalation that can have a median particle diameter of 0.5-12 μm, such as about 0.5 μm to 10 μm, preferably 1 μm to 6 μm, and more preferably about 2-4 μm. By creating inhaled particles which have a relatively narrow range of size, it is possible to further increase the efficiency of the drug delivery system and improve the repeatability of the dosing. Thus, it is preferable that the particles not only have a size in the range of 0.5 μm to 12 μm or 2 μm to 6 μm or about 0.75-4 μm but that the median particle size be within a narrow range so that 80% or more of the particles in the formulation have a particle diameter which is within ±20% of the median particle size, preferably ±10% and more preferably ±5% of the median particle size. The median particle diameter may be in the range of 0.5-8 I'm, 0.75-5 I'm, 0.5-4 I'm, 0.75-4 I'm, 0.75-3 μm 1-3 μm, or 1.5-3 μm. In some aspects, the crystalline particles (i.e., nanoparticles) of these size ranges, such as 2-4 μm, may form aggregates which are larger in size but may be measured using laser diffraction (Sympatec Software 5.6.0.0 using HRLD) to comprise particles within the above ranges.

In some aspects, the particles may be in an antisolvent and measured using a laser diffraction under mild agitation to determine the median particle diameter. In other aspects, the median particle diameter may be measured with the particles dispersed as a dry powder using a disperser system (e.g., Sympatec Rodos) using maximal shear.

The clofazimine composition may be in crystalline form. Crystalline clofazimine molecules are arranged in a highly organized, regular and repetitive structure extending in all directions. Crystalline clofazimine may have less than 10% amorphous particles. In particular embodiments, the crystalline clofazimine may have no amorphous particles. In some embodiments, the amount of amorphous clofazimine in crystalline clofazimine may be between 0-10%, 0.1-10%, 0.1-5%, 1-10%, or 1-5%. Crystalline compositions may be slow dissolving due to their highly ordered nature.

The inhalable clofazimine composition may comprise a single active ingredient (i.e., clofazimine) and, thus, may be free of any other active ingredient. The composition may be at least 90%, such as 91, 92, 93, 94, 95, 96, 97, 98, 99, or 100% clofazimine.

In particular embodiments, the inhalable clofazimine composition provided herein is essentially free of excipients and additives. In particular aspects, the present composition is free of any added excipients. The present clofazimine composition may comprise less than 10%, such as less than 5%, specifically less than 1%, particularly less than 0.1%, such as less than 0.01%, of cycodextrin, anhydrous glucose, anhydrous lactose, lactose monohydrate, mannitol, monosaccharides, disaccharides, oligosaccharides, aclidinium bromide, fumaryl diketopiperazine, magnesium stearate, cellubiose acetate, water, ethanol, isopropyl alcohol, L-leucine, Dextran, chitosan, deacetylated chitosan, ascorbic acid, stearic acid, pluronic F-68, pluronic F-127, deoxycholate, glyceryl monostearate, soybean phosphatidylcholine, poloxamer 188, Precirol ATOS, Capryol-90, lauric acid, calcium-disodium EDTA, poly(vinyl alcohol), sodium deoxycholate, sodium tripolyphosphate, lecithin, cetyl alcohol, polyvinylpyrrolidone, polycaprolactone, dipalmitoyl phosphatidylcholine, dipalmitoylphosphatidylglycerol, Lactohale 300M, Pharmatose 150M, tert-butyl alcooll, sodium deoxycholate, poly(ε-caprolactone), cholesterol, dicholoromethane, stearylamine grafted dextran, dipalmitoyl phosphatidylcholine, sodium alginate, Compritol 888, tristearin, cyclodextrin, hydroxy propyl methylcellulose, hydroxy propyl cellulose, ethyl cellulose, silica, povidone, starch, polyethylene glycol, carbomer, poly(lactic acid), poly(D,L-lactic-co-glycolic acid), hydroxypropyl cellulose, sodium carboxy methylcellulose, polymethyl methacrylate, acrolein, glycidyl methacrylate, lactides, poly(alkyl cyanoacrylate), polyanhydrides, poly(D, L-lactic-co-glycolic acid), poly(acryl) dextran, poly(acryl) starch, carrageenan, and gelatin.

Further embodiments provide methods of producing the inhalable clofazimine composition provided herein. The native clofazimine (i.e., commercially available clofazimine) may be subjected to milling, such as jet milling, particularly air jet milling, to produce the excipient-free inhalable clofazimine composition provided herein. Exemplary air jet mills which may be used in the present methods include, but are not limited to, the Aljet fluid energy mill, the Jet Pulverizer Micron-Master mill, and the Sturtevant Micronizer Jet Mill.

In one exemplary method, the native clofazimine may be micronized by using a lab-scale Aljet air jet mill (Model 00 Jet-O-Mizer™, Fluid Energy, Telford, PA), to a particle size distribution within the respirable range of 0.5-5 μm. The air jet mill may be set at a grind pressure of about 70-80 PSI, such as 75 PSI, a feed pressure of about 60-70 PSI, such as 65 PSI, and a feed rate of about 0.5-2 gram/minute, such as about 1 gram/minute. Approximately 1-20, such as 5-10, particularly 3-4.5 grams of CFZ may be milled per batch. Geometric particle size distribution for each milled batch may be assessed with a laser diffraction instrument, such as a HELOS laser diffraction instrument (Sympatec GmbH, Germany) using RODOS dispersion at 3-4 bar. Measurements may be taken every 10 msec following powder dispersion. Measurements that are between 5-25% optical density may be averaged to determine particle size distribution.

### IV. Methods of Use

In some embodiments, the present disclosure provides methods for the treatment or prevention of a pulmonary infection comprising administering the inhalable clofazimine composition provided herein. The infection may be, but is not limited to, Mycobacterium tuberculosis, multi-drug resistant M. tuberculosis, extensively drug resistant M. tuberculosis, Mycobacterium avium complex, Mycobacterium abscesses, Mycobacterium kansasii, Staphylococcus aureus, and methicillin resistant Staphylococcus aureus (MRSA). In some embodiments, the treatment may be prophylactic to subjects at risk of developing a pulmonary infection, such as subjects with a family member diagnosed with a pulmonary infection, subjects traveling to areas with high rates of pulmonary infection, or healthcare workers.

The present disclosure further provides methods of treating, reducing, or preventing a pulmonary inflammation by administering the inhalable clofazimine composition provided herein. For example, the methods may be applied to subjects with respiratory disorders such as asthma, chronic obstructive pulmonary disease, and cystic fibrosis. The respiratory disorder, in the context of present invention, includes but is not limited to asthma, emphysema, bronchitis, COPD, sinusitis, respiratory depression, reactive airways dysfunction syndrome (RADS), acute respiratory distress syndrome (ARDS), irritant induced asthma, occupational asthma, sensory hyper-reactivity, airway (or pulmonary) inflammation, multiple chemical sensitivity, and aid in smoking cessation therapy. The term “asthma” may refer to acute asthma, chronic asthma, intermittent asthma, mild persistent asthma, moderate persistent asthma, severe persistent asthma, chronic persistent asthma, mild to moderate asthma, mild to moderate persistent asthma, mild to moderate chronic persistent asthma, allergic (extrinsic) asthma, non-allergic (intrinsic) asthma, nocturnal asthma, bronchial asthma, exercise induced asthma, occupational asthma, seasonal asthma, silent asthma, gastroesophageal asthma, idiopathic asthma and cough variant asthma.

In further embodiments, methods are provided for the treatment of lung cancer, such as a reduction in lung inflammation, by administering the inhalable clofazimine composition provided herein. In another embodiment, the inhalable clofazimine composition is administered to serve as a contrast agent.

In some embodiments, treatment of a patient with micronized clofazimine may comprise modulated drug release. In some embodiments, micronized clofazimine may be formulated for slow- or delayed-release. In some embodiments, micronized clofazimine may be formulated for fast-release. In further embodiments, micronized clofazimine may be formulated for both slow and fast release (i.e., dual release profile).

In some embodiments, the present disclosure provides methods for the administration of the inhalable clofazimine composition provided herein. Administration may be, but is not limited, to inhalation of micronized clofazimine using an inhaler. In some embodiments, an inhaler is a simple passive dry powder inhaler (DPI), such as a Plastiape RSO1 monodose DPI. In a simple dry powder inhaler, dry powder is stored in a capsule or reservoir and is delivered to the lungs by inhalation without the use of propellants.

In some aspects, the required inspiratory flow rate required for the use of an inhaler may be less than 95 L/min, such as about 90 L/min, such as between about 15-90 L/min, preferably about 30 L/min. In some embodiments, efficient aerosolization of micronized clofazimine is independent of inspiratory force.

In some embodiments, an inhaler is a single-dose DPI, such as a DoseOne™ Spinhaler, Rotohaler®, Aerolizer®, or Handihaler. In some embodiments, an inhaler is a multidose DPI, such as a Plastiape RS02, Turbuhaler®, Twisthaler™, Diskhaler®, Diskus®, or Ellipta™. In some embodiments, the inhaler is Twincer®, Orbital®, TwinCaps®, Powdair, Cipla Rotahaler, DP Haler, Revolizer, Multi-haler, Twister, Starhaler, or Flexhaler®. In some embodiments, an inhaler is a plurimonodose DPI for the concurrent delivery of single doses of multiple medications, such as a Plastiape RSO4 plurimonodose DPI. Dry powder inhalers have medication stored in an internal reservoir, and medication is delivered by inhalation with or without the use of propellants. Dry powder inhalers may require an inspiratory flow rate greater than 30 L/min for effective delivery, such as between about 30-120 L/min. In some embodiments, efficient aerosolization of micronized clofazimine is independent of inspiratory force. In some embodiments, the dry powder inhaler has a flow resistance of between 0.01 kPa^{0.5} min/L and 0.06 kPa^{0.5} min/L, such as between 0.02 kPa^{0.5} min/L and 0.04 kPa^{0.5} min/L.

In some embodiments, the inhalable clofazimine is delivered as a propellant formulation, such as a HFA propellants or QNasl.

In some embodiments, the inhaler may be a metered dose inhaler. Metered dose inhalers deliver a defined amount of medication to the lungs in a short burst of aerosolized medicine aided by the use of propellants. Metered dose inhalers comprise three major parts: a canister, a metering valve, and an actuator. The medication formulation, including propellants and any required excipients, are stored in the canister. The metering valve allows a defined quantity of the medication formulation to be dispensed. The actuator of the metered dose inhaler, or mouthpiece, contains the mating discharge nozzle and typically includes a dust cap to prevent contamination.

In some embodiments, an inhaler is a nebulizer. A nebulizer is used to deliver medication in the form of an aerosolized mist inhaled into the lungs. The medication formulation be aerosolized by compressed gas, or by ultrasonic waves. A jet nebulizer is connected to a compressor. The compressor emits compressed gas through a liquid medication formulation at a high velocity, causing the medication formulation to aerosolize. Aerosolized medication is then inhaled by the patient. An ultrasonic wave nebulizer generates a high frequency ultrasonic wave, causing the vibration of an internal element in contact with a liquid reservoir of the medication formulation, which causes the medication formulation to aerosolize. Aerosolized medication is then inhaled by the patient. A nebulizer may utilize a flow rate of between about 3-12 L/min, such as about 6 L/min. In some embodiments, the nebulizer is a dry powder nebulizer.

In some embodiments, the composition may be administered on a routine schedule. As used herein, a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In some embodiments, clofazimine is administered once per day. In preferred embodiments, clofazimine is administered less than once per day, such as every other day, every third day, or once per week. In some embodiments, a complete dose of clofazimine is between 1-100 mg, such as 20-100, 50-100, 10-20, 20-40, 50-70, or 80-90 mg.

In some embodiments, clofazimine may be provided in a unit dosage form, such as in a capsule, blister or a cartridge, wherein the unit dose comprises at least 10 mg of clofazmine, such as at least 15 mg or 20 mg of clofazimine per dose. In particular aspects, the unit dosage form does not comprise the administration or addition of any excipient and is merely used to hold the powder for inhalation (i.e., the capsule, blister, or cartridge is not administered). In some embodiments, clofazimine may be administered in a high emitted dose, such as at least 10 mg, preferably at least 15 mg, even more preferably 20 mg. In some embodiments, administration of micronized clofazimine results in a high fine particle dose into the deep lung such as greater than 5 mg. Preferably, the fine particle dose into the deep lung is at least 10 mg, even more preferably at least 15 mg. In some aspects, the fine particle dose is at least, 50%, such as at least 60, 65, 70, 75, or 80% of the emitted dose.

In some embodiments, changes in pressure drop across the device result in a change in emitted dose. In some embodiments, changes in pressure drop across the device of 3 kPa, such as from 4 kPa to 1 kPa, result in a reduction of emitted dose of less than 25%, such as 24%, 23%, 22%, 21%, 20%, 19%, 18%, 17%, 16%, 15%, 14%, 13%, 12%, 11%, 10%, 9%, 8%, 7%, 6%, 5% or less. In some embodiments, changes in inhalation pressure drop across the device result in a change in fine particle dose. In some embodiments, changes in inhalation pressure drop across the device of 3 kPa, such as from 4 kPa to 1 kPa result in a reduction of fine particle dose of less than 15%, such as 14%, 13%, 12%, 11%, 10%, 9%, 8%, 7%, 6%, 5% or less.

In some embodiments, the dissolution rate of clofazimine is measured. In some embodiments, crystalline clofazimine has a slow dissolution rate. In some embodiments, the dissolution rate of clofazimine is such that no more than 30%, such as less than 25, 20, 15, or 10%, of the clofazimine by mass dissolves in dissolution media within 15 minutes of addition. In some embodiments, the dissolution media is Phosphate Buffered Saline pH 7.4+0.2% polysorbate 80.

In some embodiments, clofazimine is internalized by J774.A1 macrophage cultures. In some embodiments, the clofazimine is crystalline. In some embodiments the clofazimine is micronized. In some embodiments, micronized crystalline clofazimine particles are internalized by J774.A1 macrophage cultures. In further embodiments, the rate of internalization of the particles by macrophages is high, such as greater than 80% internalization after 8 hours of incubation. In some embodiments, macrophages transform the clofazimine into a different crystalline-like form. In some embodiments, change in crystalline form of clofazimine is detected by a fluorescence shift. In some embodiments, the fluorescence shift is from around 590 nm to around 660 nm. In some embodiments, the fluorescence shift occurs within a short time. In some embodiments, the fluorescence shift occurs within 1 week, such as in 7 days, 6 days, 5 days, 4, days 3 days, 2 days, or within 24 hours.

In some embodiments, the treatment methods provided herein may further comprise administering at least a second therapeutic agent. The second agent may be, but is not limited to, bedaquilline, pyrazinamide, nucleic acid inhibitors, protein synthesis inhibitors, and cell envelope inhibitors. The group protein synthesis inhibitors may include, but are not limited to, linezolid, clarithromycin, amikacin, kanamycin, capreomycin, and streptomycin. The group cell envelope inhibitors may include, but are not limited to, ethambutol, ethionamide, thioacetizone, isoniazid, imipenem, clavulanate, cycloserine, terizidone, amoxicillin, and prothionamide. The group nucleic acid inhibitors may include, but are not limited to, rifampicin, rifabutin, rifapentine, 4-aminosalicylic acid, moxifloxacin, ofloxacin, and levofloxacin. In some embodiments, the second therapeutic agent may be clofazimine. Other exemplary agents include but are not limited to vancomycin, tobramycin, ciprofloxacin, fosfomycin, and rifaximin. The combination therapies may be administered simultaneously, sequentially, or separately.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### IV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Materials and Methods

Micronization of clofazimine: A lab-scale Aljet air jet mill (also known as a Model 00 Jet-O-Mizer™, Fluid Energy, Telford, PA) was used to micronize clofazimine (Sigma; Lot: SLBL8945V) to a particle size distribution within the respirable range of 0.5-5 μm. Nitrogen gas at a grinding pressure of 75 PSI and a feed pressure of 65 PSI was used, coupled with a solid material feed rate of 1 gram/min. Geometric particle size distribution for each milled batch was assessed with a HELOS laser diffraction instrument (Sympatec GmbH, Germany) (Sympatec Software 5.6.0.0 using HRLD) using RODOS dispersion at 3 bar. Measurements were taken every msec following powder dispersion. Measurements that were between 5-25% optical density were averaged to determine particle size distribution.

Scanning electron microscopy of micronized clofazimine: To analyze morphology of milled clofazimine, samples were mounted on aluminum SEM stubs and sputter coated with 12 nm of platinum/palladium (Pt/Pd) using a Cressington sputter coater 208 HR (Cressington Scientific Instruments Ltd., Watford, UK). Imaging was performed using a Zeiss Supra 40VP SEM (Carl Zeiss Microscopy GmbH, Jena, Germany). Undispersed particles and particles dispersed using the RODOS disperser at 3 bar were examined.

X-ray diffraction crystallography and Differential Scanning calorimetry: The presence of crystallinity and polymorph transformation in milled clofazimine was determined using X-ray powder diffraction (XRD) and differential scanning calorimetry (DSC). One-dimensional diffractograms of unmilled and milled clofazimine powder were obtained using a Rigaku MiniFlex 600 II (Rigaku Corporation, Tokyo, Japan), controlled by Rigaku Guidance software and set to a target radiation of copper at 40 KV voltage and 40 mA current. Diffractograms were analyzed using Jade (Ragaku Corporation, Tokyo, Japan). Thermograms of unmilled and milled clofazimine were obtained using an Auto Q20 DSC controlled by the TA Advantage Software and equipped with a RCS40 (TA Instruments-Waters LLC, New Castle, DE, USA) refrigerated cooling system with nitrogen purge of 50 mL/min. Approximately 4 mg of each sample were loaded in standard DSC pans (DSC Consumables Inc., Austin, MN, USA) and were crimped using a Tzero sample press (TA Instruments-Waters LLC, New Castle, DE, USA). Samples were heated at a rate of 5° C./min from 30° C. to 300° C.

Specific surface area analysis of excipient-free milled clofazimine: The specific surface area of milled clofazimine was assessed using a Monosorb gas adsorption unit (Quantachrome Instruments). Three samples were loaded into glass measurement cells and allowed to outgas under helium at 80° C. for 18 hours. Using single point Brauner Emmett Teller (BET) method and 30 mol fraction nitrogen in helium as the adsorbate, the surface area of each sample was calculated. To determine the specific surface area, the surface area was divided by the sample weight after outgassing.

Density analysis of excipient-free milled clofazimine: To assess bulk and tapped density of excipient-free milled clofazimine, a glass test tube was calibrated to 0.25 mL using a pipette. The tube was filled to volume corresponding to 2-3 mL calibration mark and then weighed to obtain the bulk density. The tube was then tapped 10 times and the volume was remeasured to obtain the tapped density.

Angle of repose analysis of excipient-free milled clofazimine: To assess the angle of repose of excipient-free milled clofazimine, approximately 500-800 mg of milled clofazimine was poured from a funnel placed approximately 4.5 cm above a hollow, open cylinder with a radius of 1.25 cm and a height of 1.2 cm. The height of the cone resulting from the poured powder was determined using the image analysis software Image J. Three cone heights were taken and averaged for the final angle of repose determination. The angle of repose was calculated according to equation below:

$$tan ( α ) = \frac{height}{0.5 base}$$

Determination of aerosolization performance of excipient-free milled clofazimine: In vitro aerodynamic performance testing was performed utilizing a Model 7 low-resistance Monodose RS01 DPI and a high-resistance Monodose RS01 DPI, from Plastiape S.p.a (Osnago, Italy). Size 3 hydroxypropyl methylcellulose (HPMC) capsules were provided by Capsugel Inc. (Morristown, New Jersey, USA). The resistance of the low-resistance RS01 Monodose DPI used in the cascade impaction studies was determined using a dosage sampling unit according to apparatus B of USP chapter 601, and was calculated to be 0.021 kPa^{0.5} min/L. The resistance of the high-resistance RS01 Monodose DPI used in the cascade impaction studies was reported to be 0.036 kPa^{0.5} min/L (Elkins, Anderson et al. 2014). Cascade impaction studies for milled clofazimine were performed on the Next Generation Impactor (NGI) (MSP Corporation, MN, USA). Stage 1-7 cut-off diameters were determined using equation 1 and MOC cut-off diameters were determined using equation 2.

$${D}_{50 , Q} = {{D}_{50 , Q n} ( \frac{{Q}_{n}}{Q} )}^{X} \qquad Eq ( 1 )$$

$${D}_{80 , Q} = 0.14 {( \frac{{Q}_{n}}{Q} )}^{1.36} \qquad Eq ( 2 )$$

where D_{50,Q} is the cutoff diameter at the flow rate, Q, and the subscript, n, refers to the archival reference value for Qₙ=60 L/min, and the values for the exponent, x, were determined by the archival NGI stage cut size-flow rate calculations, as determined by Marple et al. To reduce particle bounce and re-entrainment, the NGI plates were coated with 1% (v/v) silicon oil in hexane and allowed to dry. To determine the influence of particle size on aerodynamic performance of milled clofazimine, analysis conducted using the low-resistance RS01 DPI was performed on milled particles obtained specifically from the second milled batch. Two different populations of particles were tested: those with a median geometric diameter (D₅₀) of 2.69 μm (CFZ2.69 μm), derived from the cyclone unit of the jet mill (FIG. 1) and milled particles with a D₅₀ of 1.81 μm (CFZ1.81 μm), derived from the collection vessel unit of the jet mill (FIG. 1). Cascade impaction was performed on these samples at a 4 kPa pressure drop (equivalent to 93 L/min on the low-resistance RS01 device and equivalent to 55.6 L/min on the high-resistance RS01 device) at a duration of time sufficient to draw 4 liters of air through the apparatus (equivalent to 2.6 seconds on the low-resistance RS01 device and 4.3 seconds on the high-resistance RS01 device). To determine the flow rate dependency of milled clofazimine dispersion, cascade impaction was also performed on CFZ1.81 μm particles at a 1 kPa pressure drop through the device (equivalent to 47 L/min) for a duration of 5.1 seconds. To compare performance of milled clofazimine aerosolized from the low resistance RS01 device versus the high resistance RS01 device, clofazimine samples from a milled batch with a volume median particle size of 2.44 μm (as measured using a Sympatec laser diffraction unit with RODOS dry dispersion at 4 bar pressure and analyzed using HELOS software version 5.6.0.0 with an HRLD model to determine particle size distribution) were utilized. The resultant dispersed powder was collected from the capsule, the inhaler, the adapter, the induction port, stages 1-7 and the micro-orifice collector (MOC) by washing with ethanol or isopropyl alcohol. The drug mass in each sample was quantified by measuring the UV-absorbance at a wavelength of 480 nm using a Tecan Infinite M200 PRO multimode microplate reader (Tecan Systems, Inc., San Jose, CA, USA). The emitted fraction (EF) was calculated as the total drug emitted from the device as a percentage of the total mass of drug collected. The fine particle (<5 μm) fraction (FPF5_{5μm/EF}) and fine particle (<3 μm) fraction (FPF3_{3μm/EF}) corresponded to the percentage of the emitted dose predicted to have the aerodynamic diameter below 5 μm and 3 μm. The FPF5_{5μm/EF}, and FPF3_{3μm/EF} values were interpolated from a graph with the cumulative percentage of the emitted dose deposited downstream from an NGI stage as the ordinate and the particle cutoff size of that stage as the abscissa. For each sample, the mass median aerodynamic diameter (MMAD), which represents the mass-based median point of the aerodynamic particle size distribution (APSD), and geometric standard deviation (GSD), which represents the spread of the APSD, were determined by plotting the cumulative percentage of mass less than the stated aerodynamic size cut (expressed as Probits) against the aerodynamic diameter (log scale). Distributions were log normal. A linear regression was performed to determine the aerodynamic diameters corresponding to the 50% percentile (Probit 5) to determine the MMAD, and the aerodynamic diameters corresponding the 15.87% percentile (Probit 4) and 84.13% percentile (Probit 6) to calculate the GSD.

Determination of aerosolization performance of milled clofazimine blended with lactose: Milled clofazimine comprising a median particle size of 2.44 μm, as measured using a Sympatec laser diffractor with RODOS dispersion at 4 bar pressure, was blended with Inhalac 230 lactose (Meggle Pharma), reported to have a median particle size of 70-110 μm. 135 mg of milled clofazimine was mixed with 15 mg of lactose via a process of spatulation inside a glass scintillation vial. The lactose was added first, and CFZ was incorporated via a process of geometric dilution. 5 samples were taken to assess blend uniformity, and the average potency of clofazimine in the blend was 86.5% w/w. In vitro aerodynamic performance testing was performed utilizing a Model 7 low-resistance Monodose RSO1 DPI, from Plastiape S.p.a (Osnago, Italy). Size 3 hydroxypropyl methylcellulose (HPMC) capsules were provided by Capsugel Inc. (Morristwon, New Jersey, USA). The resistance of the RSO1 Monodose DPI used in the cascade impaction studies was determined using a dosage sampling unit according to apparatus B of USP chapter 601, and was calculated to be 0.021 kPa^{0.5} min/L. Cascade impaction studies for milled clofazimine were performed on the Next Generation Impactor (NGI) (MSP Corporation, MN, USA). Stage 1-7 cut-off diameters were determined using equation 1 and MOC cut-off diameters were determined using equation 2.

$${D}_{50 , Q} = {{D}_{50 , Q n} ( \frac{{Q}_{n}}{Q} )}^{X} \qquad Eq ( 1 )$$

$${D}_{80 , Q} = 0.14 {( \frac{{Q}_{n}}{Q} )}^{1.36} \qquad Eq ( 2 )$$

where D_{50,Q} is the cutoff diameter at the flow rate, Q, and the subscript, n, refers to the archival reference value for Qₙ=60 L/min, and the values for the exponent, x, were determined by the archival NGI stage cut size-flow rate calculations, as determined by Marple et al. To reduce particle bounce and re-entrainment, the NGI plates were coated with 1% (v/v) silicon oil in hexane and allowed to dry. To determine the influence of particle size on aerodynamic performance of milled clofazimine, analysis was performed on milled particles obtained specifically from the second milled batch. Two different populations of particles were tested: those with a median geometric diameter (D₅₀) of 2.69 μm (CFZ2.69 μm), derived from the cyclone unit of the jet mill (FIG. 1) and milled particles with a D₅₀ of 1.81 μm (CFZ1.81 μm), derived from the collection vessel unit of the jet mill (FIG. 1). Cascade impaction was performed on these samples at a 4 kPa pressure drop (equivalent to 93 L/min on the low-resistance RS01 device) at a duration of time sufficient to draw 4 liters of air through the apparatus (2.6 seconds). The resultant dispersed powder was collected from the capsule, the inhaler, the adapter, the induction port, the pre-separator, stages 1-7 and the micro-orifice collector (MOC) by washing with isopropyl alcohol. The drug mass in each sample was quantified by measuring the UV-absorbance at a wavelength of 480 nm using a Tecan Infinite M200 PRO multimode microplate reader (Tecan Systems, Inc., San Jose, CA, USA). The emitted fraction (EF) was calculated as the total drug emitted from the device as a percentage of the total mass of drug collected. The fine particle (<5 μm) fraction (FPF5_{5μm/EF}) and fine particle (<3 μm) fraction (FPF3_{3μm/EF}) corresponded to the percentage of the emitted dose predicted to have the aerodynamic diameter below 5 μm and 3 μm. The FPF5_{5μm/EF}, and FPF3_{3μm/EF} values were interpolated from a graph with the cumulative percentage of the emitted dose deposited downstream from an NGI stage as the ordinate and the particle cutoff size of that stage as the abscissa. For each sample, the mass median aerodynamic diameter (MMAD), which represents the mass-based median point of the aerodynamic particle size distribution (APSD), and geometric standard deviation (GSD), which represents the spread of the APSD, were determined by plotting the cumulative percentage of mass less than the stated aerodynamic size cut (expressed as Probits) against the aerodynamic diameter (log scale). Distributions were log normal. A linear regression was performed to determine the aerodynamic diameters corresponding to the 50% percentile (Probit 5) to determine the MMAD, and the aerodynamic diameters corresponding the 15.87% percentile (Probit 4) and 84.13% percentile (Probit 6) to calculate the GSD.

Macrophage uptake of milled clofazimine: J774.A1 murine macrophages were cultured in Dulbecco's Modified Eagle Medium (DMEM) supplemented with 10% Fetal Bovine Serum (FBS), 1% penicillin, and 1% streptomycin. Cells were maintained at 5% CO2 at 37° C. Passaging was performed before cells reached 80% confluency.

To determine toxicity of milled clofazimine as compared to solubilized clofazimine, an MTT assay was performed. J774.A1 cells were plated in a 96 well plate at 104 cells/well in replicates of six and allowed to grow for 24 hours. Varying concentrations (5 I'M, 10 I'M, 20 I'M) of solubilized or milled particles were added to the cells, and cells were incubated for 24 hours. Solubilized clofazimine treatments were made by dissolving clofazimine in DMSO and diluting accordingly from a stock concentration. No more than 0.4% of DMSO was added to cells to reduce any toxic effect of DMSO on cells. Milled clofazimine treatments were made by suspending milled particles in PBS, sonicating for 5 minutes to ensure dispersion, and diluting accordingly. After 24 hours of exposure, drug treatments were removed and cells were incubated with an MTT reagent solution (0.5 mg/mL in phenol-free medium) for 1 hour at 37° C. and 5% CO2. All but 25 μL of the MTT reagent was then aspirated and 50 μl of DMSO were added to solubilize cells. The plate was read with spectrophotometer (Infinite M200, Tecan) at 540 nm. The absorbance of the treated cells was normalized with the positive controls of cells treated with PBS or DMSO only.

To assess the phagocytosis rate of milled clofazimine, J774.A1 macrophages were imaged at various time points following drug exposure. Cells were seeded at 3×10⁵ cells 35 mm glass-bottomed dish and were allowed to grow for 24 hours, at which point D₅₀1.90 μm clofazimine particles (derived from collection vessel region of Aljet mill) and D₅₀ 2.83 μm clofazimine particles (derived from cyclone region of Aljet mill) were added. Drug treatments were prepared as described for the MTT assay. All treatment groups were added to the macrophages at a concentration of 20 μg/mL. Brightfield images were taken on an EVOS XL Core Imaging System (Thermo Fisher Scientific; Waltham, MA) at 40× magnification. Time point images were taken at 0.5 hours, 1 hour, 2 hours, 4 hours, 6 hours, 8 hours, and 24 hours of drug exposure. At least 6 images were obtained at each time point. A manual count of clofazimine particles inside of the cells and outside of the cells was performed to determine particle uptake rate, with at least 540 cells counted at each time point. A curve was fitted to the data using Excel (Microsoft Corporation).

Flow cytometric quantification of clofazimine: To quantify macrophage uptake of clofazimine and assess for the intracellular bio-transformation of milled clofazimine to liquid crystals, flow cytometry was performed. Experimental set-up for flow cytometry was similar to the microscopy experiment. Treatment groups consisted of solubilized clofazimine, unmilled clofazimine, D₅₀1.90 μm clofazimine particles (averaged size; derived from collection vessel region of Aljet mill) and D₅₀2.83 μm clofazimine particles (averaged size; derived from cyclone region of Aljet mill), as well as control groups that contained either PBS or DMSO. After 24 hours of drug exposure, drug-containing medium was removed and cells were suspended in 1 mL of PBS for analysis. An Accuri SORP Flow Cytometer (BD, Franklin Lakes, NJ, USA) equipped with a 551-nm laser was used to analyze cells based on the presence of engulfed clofazimine. Sorting was performed at both a bandpass of 610/20 bp (corresponding to triclinic form of clofazimine) and 660/20 bp (corresponding to liquid crystal form of clofazimine). A cytogram based on the forward angle light scatter (FSC) versus the right angle side scatter (SSC) was used to eliminate aggregates, debris, and dead cells before fluorescence was detected. All gating and analysis was performed in triplicate on at least 10,000 cells. Sample acquisition was performed on FACSDiva v6.1.3 (BD, Franklin Lakes, NJ, USA). Sample analysis was performed on FlowJo (FlowJo, LLC, Ashland, OR).

Dissolution of milled clofazimine: Dissolution study of milled clofazimine utilized the NGI with a modified impactor stage to allow for the collection of aerodynamically separated particles, which was then placed inside a USP Apparatus II (paddle) dissolution bath. In order to quantify the dissolution of the poorly water soluble clofazimine, PBS containing 0.2% polysorbate 80 was used as the dissolution medium. Prior to the dissolution study, the saturation solubility of clofazimine in PBS+0.2% polysorbate 80 was determined by placing an excess of milled clofazimine into the medium and placing in a MaxQ 4450 Shaker (Thermo Scientific, Waltham, MA, USA) at 75 RPM and 37° C. for 24 hours, after which shaking was stopped and samples settled for 48 hours at 37° C. An aliquot was drawn from the supernatant and was assessed with a spectrophotometer (Infinite M200, Tecan). To assess dissolution of milled clofazimine, 9 mg of drug was loaded into a capsule and actuated into an NGI. Stage 5 (corresponding to a 0.75 μm aerodynamic particle size cut off at 93 L/min) was replaced with the modified NGI dissolution stage. After dispersion into the NGI, the powder in the modified stage was covered with a 90-mm diameter, 0.05 μm pore-size Whatman® polycarbonate filter (GE Healthcare Life Sciences, Chicago, IL, USA) cut to size, sealed with the corresponding O-ring, and placed in Varian VK2000 Dissolution Bath (Agilent Technologies, Santa Clara, CA, USA) with paddle apparatus. The dissolution vessel contained 300 mL of pre-warmed dissolution medium and the paddle was set 10 mm away from the stage. Rotation was set at 75 RPM and temperature was set at 37° C. 3 mL samples were taken over the course of 24 hours, and replaced with new medium. After the study conclusion, the drug remaining in the modified NGI stage was washed with ethanol. Samples were analyzed using fluorescence (480 nm excitation, 580 nm emission), to enable detection of minute quantities of drug.

TABLE 1
Target profile of CFZ DPI formulation for pulmonary delivery

| Barrier: | Method to address barrier: |
|---|---|
| High-dose needed for efficacy | Develop excipient-free formulation |
| Must target infection site in | Ensure MMAD < 3 μm |
| peripheral lung | |
| TB accompanied by lung | Aerosolization performance |
| remodeling and altered lung | independent of patient inspiratory flow |
| function, and affects pediatric | rate |
| patients | |
| TB predominates in low- | Utilize cost-effective manufacturing |
| resource countries | and delivery system |

Statistical analysis: The statistical significance of experimental results was assessed using Analysis of Variance (ANOVA) in Excel (Microsoft Corporation) with Tukey HSD post-hoc analysis where required. Alpha level was set at 0.05.

### Example 2—Characterization of Micronized Clofazimine

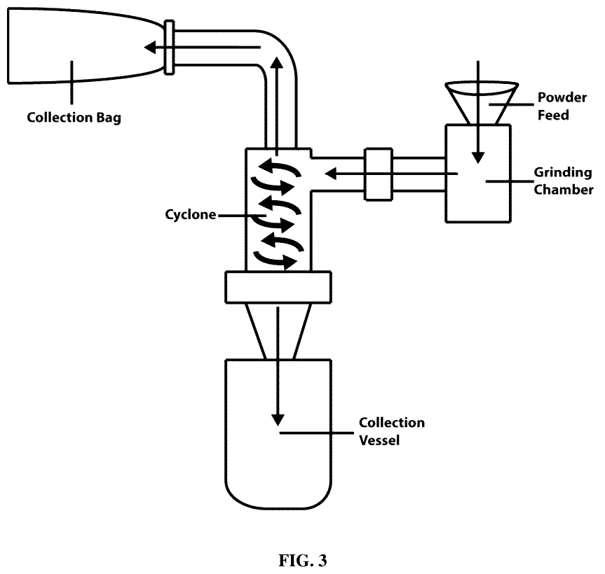

Jet milling of clofazimine according to the described conditions resulted in a particle size distribution (PSD) within the respirable range, with the size of the particles varying according the area of the jet mill from which they were collected (FIG. 4 and Table 2). The average percent yield of the milling process was 48.34%±4.82%. The cyclone and collection vessel portions (FIG. 3) of the mill exhibited the greatest yield, with an average of 71.96%±4.93% and 13.35%±8.32% respectively and were therefore selected for further studies.

TABLE 2
Averaged (n = 3) particle size distributions of milled clofazimine

| | X₁₀ (μm) | X₁₀ (μm) | X₁₀ (μm) | Span |
|---|---|---|---|---|
| Unprocessed clofazimine | 1.55 | 7.90 | 21.96 | 2.62 |
| Milled clofazimine: | 0.95 | 2.83 | 6.11 | 1.83 |
| Cyclone area of Aljet mill | | | | |
| Milled clofazimine: | 0.76 | 1.90 | 3.95 | 1.68 |
| collection vessel area of | | | | |
| Aljet mill | | | | |

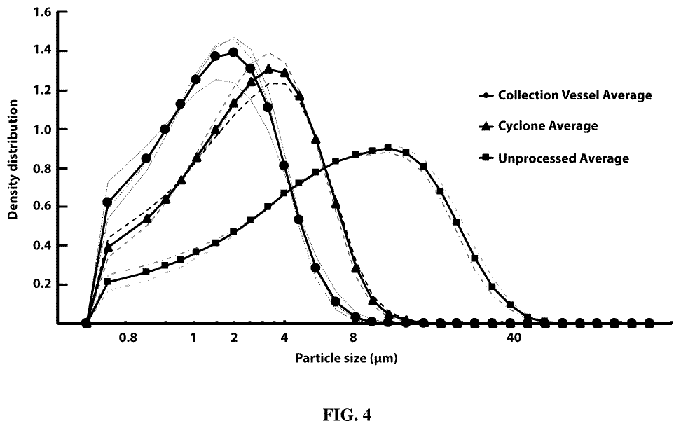

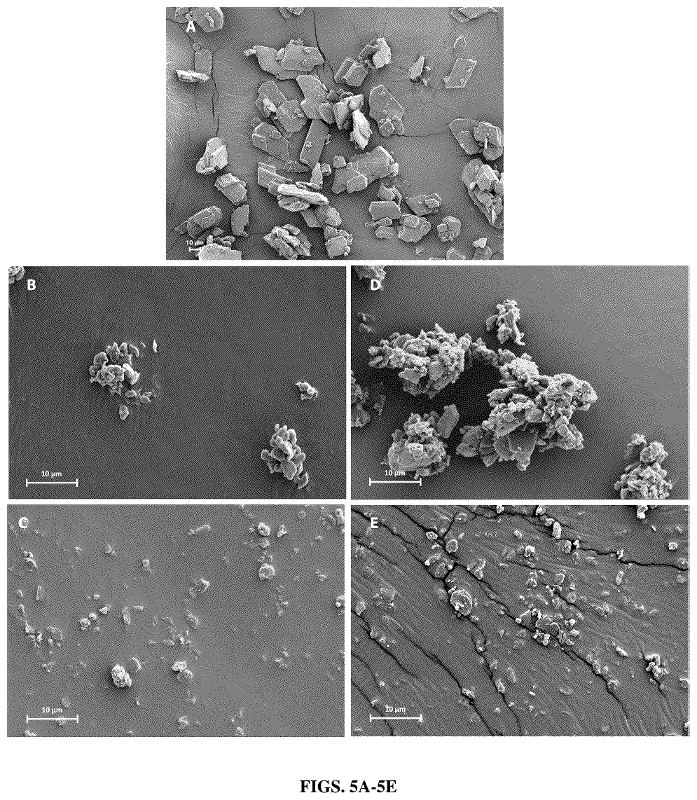

SEM images revealed unmilled clofazimine exhibited a tabular crystal habit which is maintained upon milling, and highly agglomerated milled particles that disperse into primary particles upon dispersion with RODOS at 3 bar pressures (FIG. 5).

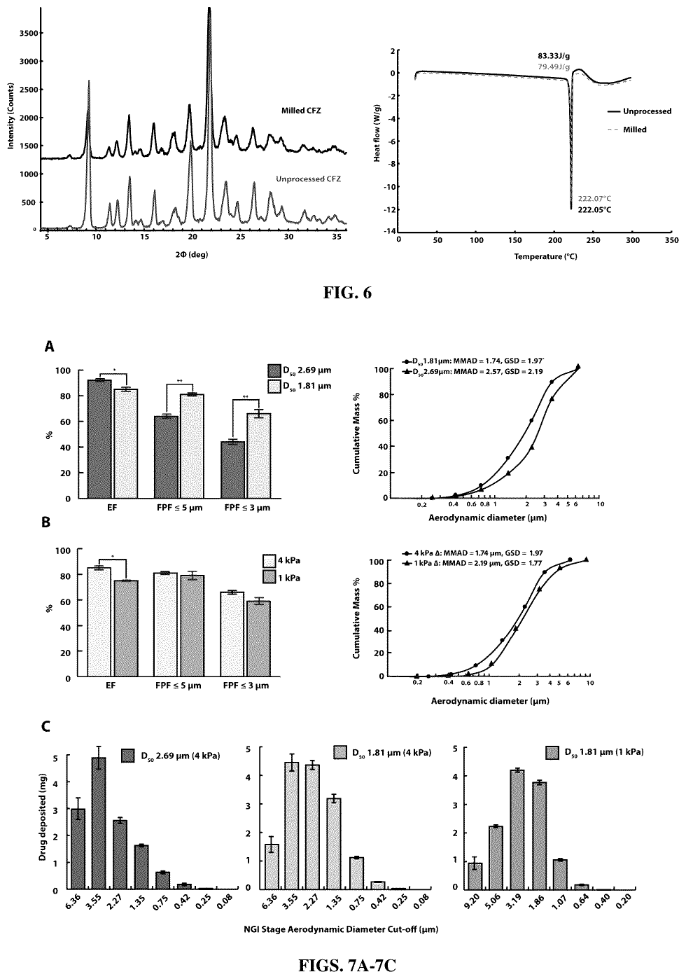

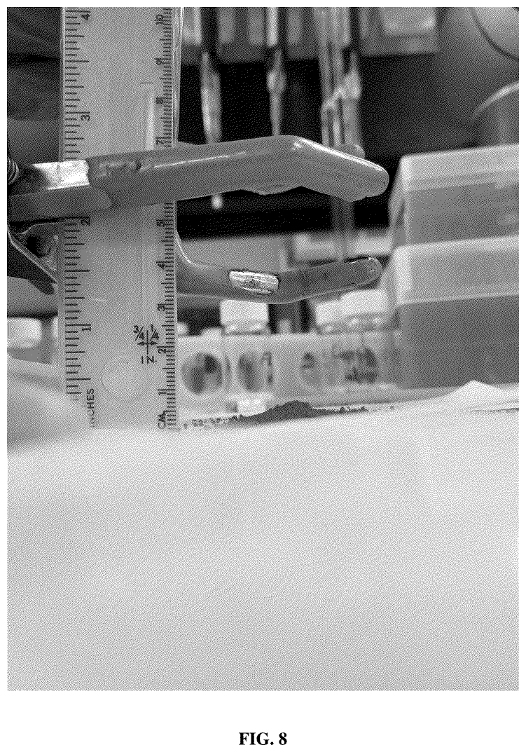

XRD and DSC analysis revealed no detectable changes in crystallinity (FIG. 6). Diffractograms of unmilled clofazimine and milled clofazimine demonstrated identical peak position. Thermograms of both unmilled and milled showed a single endothermic event at 222° C.

Surface area analysis using single-point BET method showed an average specific surface area of 2.149 m²/g. Density analysis of excipient-free milled clofazimine demonstrated an average bulk density of 0.09 g/mL and an average tapped density of 0.14 g/mL. This results in a compressibility index of 33.97 and a Hausner ratio of 1.5. Angle of repose analysis of excipient-free milled clofazimine demonstrated an angle of repose of 22.82 deg.

To assess aerosolization performance of excipient-free milled clofazimine, 20 mg of pure milled clofazimine was aerosolized from the low resistance RS01 Monodose DPI without the need for additional excipients or processing steps. The performance of D₅₀2.69 μm and D₅₀1.81 μm samples were compared (FIG. 7A, 7C). A 0.88 μm reduction in median geometric particle diameter resulted in an 8% smaller EF (P=0.014), a 27% higher FPF5_{5μm/EF} (P=0.0006) and a 50% higher FPF3_{3μm/EF} (P=0.0006) (FIG. 7A). MMAD was closely correlated to geometric median diameter (FIG. 7A), with D₅₀2.69 μm and D₅₀1.81 μm having an MMAD of 2.57 μm and 1.74 μm respectively. Sample D₅₀1.81 μm was also tested at reduced pressure drop (FIG. 7B, 7C). Reduction in device pressure drop from 4 kPa (93 L/min) to 1 kPa (47 L/min) resulted in a 12% decrease in EF (P=0.002) and a 5% decrease in FPF5_{5μm/EF} (P=0.290), and a 11% decrease in FPF3_{3μm/EF} (P=0.054), though these were not statistically significant (FIG. 7B). Though samples had the same geometric median diameter, when the device pressure drop was decreased, the MMAD increased from 1.74 μm±0.08 μm to 2.19 μm±0.08 μm (P=0.002) (FIG. 7B).

To assess the influence of device resistance on aerosolization performance of excipient-free milled clofazimine 20 mg of pure milled clofazimine (median particle size of 2.44 μm) was aerosolized from the low resistance RSO1 Monodose DPI or high resistance Monodose DPI. Using the low resistance RSO1, samples from this batch of milled clofazimine were found to have an EF of 90.19%, a FPF5_{5μm/EF} of 63.45%, and a FPF3_{3μm/EF} of 44.59%, while using the high resistance RSO1 device the EF was 83.52%, FPF5_{5μm/EF} was 68.75%, and a FPF3_{3μm/EF} of 48.90%.

To assess aerosolization performance of milled clofazimine blended with inhalation-grade lactose, 20 mg of a clofazimine-lactose blend containing approximately 90% clofazimine was aerosolized from the low resistance RSO1 Monodose DPI, and compared against performance of excipient-free milled clofazimine from the same milling batch. Using the low resistance RSO1, excipient-free milled clofazimine from this batch of milled clofazimine was found to have an EF of 90.19%, a FPF5_{5μm/EF} of 63.45%, and a FPF3_{3μm/EF} of 44.59%. Clofazimine from the same batch blended with lactose in an approximately 90:10 ratio was found to have a EF of 92.69%, a FPF5_{5μm/EF} of 69.44%, and a FPF3_{3μm/EF} of 50.91%.

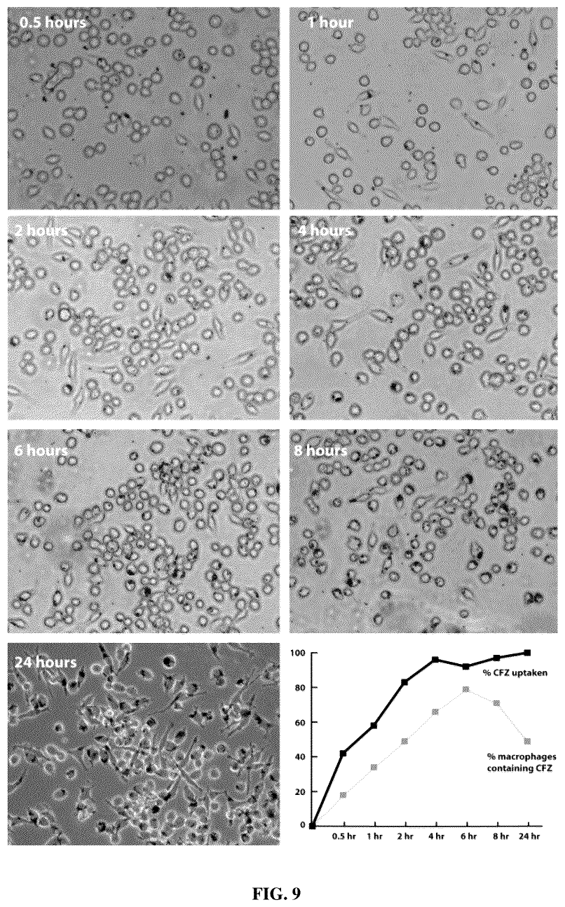

To assess Macrophage uptake of milled clofazimine, J774.A1 macrophages were incubated with either D₅₀1.90 μM or D₅₀2.83 μm clofazimine. Preliminary experiments qualitatively indicated that, there was no difference in the macrophage phagocytosis of D₅₀1.90 μM clofazimine particles (averaged size; derived from collection vessel region of Aljet mill) and D₅₀2.83 μm clofazimine particles (averaged size; derived from cyclone region of Aljet mill). Manual counts performed on D₅₀1.90 μm CFZ particles were therefore only considered in the determination of the overall macrophage phagocytosis rate of milled clofazimine. Macrophage uptake of milled clofazimine occurred rapidly, with the majority (96-97%) of milled particles internalized by 4 to 8 hours following drug exposure (FIG. 9) Transformation of internalized particles to a distinctive needle-like morphology, indicative of liquid crystal formation, was noted 24 hours after initial exposure. The largest number of macrophages containing CFZ was noted 6 hours after drug exposure, with decreasing numbers of drug-containing cells at later time points likely due to continuing cell division.

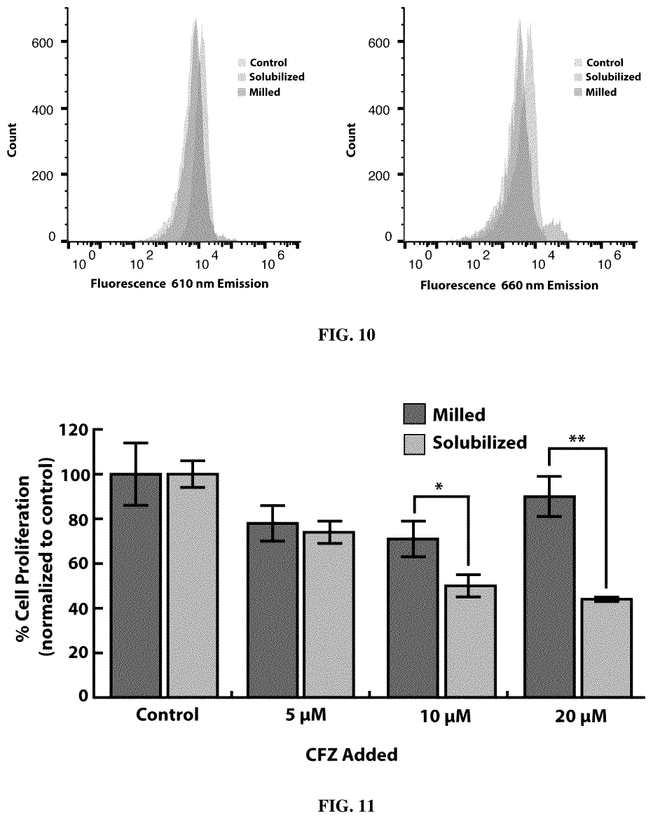

Flow cytometry analysis of J774.A1 macrophages exposed to different clofazimine treatments for 24 hours revealed that clofazimine uptake and intracellular biotransformation to clofazimine liquid crystals was dependent upon the type of treatment applied. Based upon fluorescence at 610 nm emission (specific for crystalline clofazimine), of the 10,000 cells analyzed, 2.48%±0.55% of cells exposed to solubilized clofazimine contained drug (Table 3 and FIG. 10). A higher percentage of cells exposed to milled D₅₀2.83 μm clofazimine exhibited fluorescence at this wavelength, with 4.11%±0.11% (P=0.001) of cells containing drug. Compared to the solubilized drug treatment, cells exposed to milled D₅₀1.90 μm clofazimine also contained a higher number of fluorescent cells (3.24%±0.42%), but this difference was not significant (P=0.063). Analyzing cells based upon fluorescence at 660 nm emission, which is indicative of clofazimine biotransformation by macrophages to liquid crystals, revealed significant differences between solubilized clofazimine treatments and milled clofazimine treatments (FIG. 10). Only 0.21%±0.06% of cells exposed to solubilized clofazimine exhibited fluorescence (Table 4). 5.83%±0.12% of cells exposed to milled D₅₀2.83 μm clofazimine and 5.58%±0.19% of cells exposed to milled D₅₀1.90 μM clofazimine exhibited fluorescence, which was significantly higher than the solubilized treatment group (P=0.001). There were no significant differences between the two milled treatment groups (P=0.158).

Proliferation was assessed with an MTT assay. Compared to control, the MTT assay revealed a significant decrease in cell proliferation/viability after 24 hours with increasing concentrations of solubilized clofazimine and (P=1.92×10−14) (FIG. 11). Compared to control, the MTT assay revealed a decrease in cell proliferation/viability with increasing concentrations of solubilized CFZ (FIG. 11). A significant difference in cell viability between the solubilized and milled CFZ treatment groups was noted at 10 μm (P=0.045) and 20 μm (P=0.001) drug concentrations.

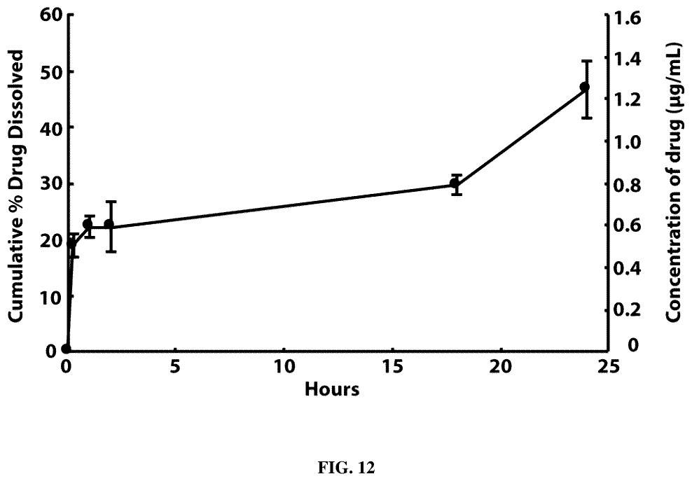

To evaluate the dissolution of milled clofazimine, the intrinsic solubility of milled clofazimine was measured in PBS pH 7.4+0.2% polysorbate 80 to be 10.9 μg/mL. Milled clofazimine showed a low degree of dissolution, with 23% of Dₐₑ0.75 μm clofazimine particles dissolved within 2 hours, with 48% dissolution achieved at 24 hours and a final concentration of drug at 1.25 μg/mL±0.14 μg/mL at 24 hours (FIG. 12).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Arbiser et al., J. Am. Acad. Dermatol. 1995, 32 (2), 241-247.
Baik et al., PLoS One 2012, 7 (10), 474-94.
Baik et al., Antimicrob. Agents Chemother. 2013, 57 (3), 1218-30.
Bolla et al., Crystal Growth & Design 2012, 12 (12), 6250-6259.
Bloom, Tuberculosis: pathogenesis, protection, and control. ASM Press: Washington, D C, 1994.
Cavanaugh et al., J. Antimicrob. Chemother. 2017, 1678-1687.
Cholo et al., J. Antimicrob. Chemother. 2012, 67 (2), 290-298.
Clofazimine. Tuberculosis 2008, 88 (2), 96-99.
Clofazimine. Aug. 1, 2009 ed.; American Society of Health-System Pharmacists, Inc: Bethesda, Maryland.
Cunningham, Dear doctor letter regarding distribution changes for lamprene, 2004.
Diacon et al., Am. J. Respir. Crit. Care Med. 2015, 191 (8), 943.
Elkins et al., The open respiratory medicine journal 2014, 8: 8.
Gangadharam et al., Tuber. Lung Dis. 1992, 73 (4), 192-199.
Global Tuberculosis Report 2016. World Health Organziation: 2016; pp 214-214.
Fisher, Milling of Active Pharmaceutical Ingredients. Encyclopedia of Pharmaceutical Technology, Third Edition, Taylor & Francis Online: 2013, 2339-2351.
Holdiness, Clinical Pharmacokinetics of Clofazimine. Clin. Pharmacokinet. 1989, 16 (2), 74-85.
Kemper et al., Ann. Intern. Med. 1992, 116 (6), 466-472.
Keswani et al., Mol. Pharm. 2015, 12 (7), 2528-2536.
Lamprene®, [package insert]. 2006.
Lindholm-Levy et al., Tubercle 1988, 69 (3), 179-186.
Louey, Journal of Aerosol Science 2006, 37(11): 1520-1531.
Mangal et al., Pharmaceutical research 2018, 35(2): 28.
Nix et al., Tuberculosis 2004, 84 (6), 365-373.
Rastogi et al., Curr. Microbiol. 1996, 33 (3), 167-175.
Reddy et al., Antimicrob. Agents Chemother. 1996, 40 (3), 633-36.
Russell, Nature Reviews Microbiology 2007, 5 (1), 39-47.
Schön et al., The international journal of tuberculosis and lung disease 2011, 15 (4), 502.
Sabnis, Inhaled Clofazimine Delivery for the Treatment of Pulmonary Tuberculosis. Masters, Creighton University, 2015.
Shafran et al., N. Engl. J. Med. 1996, 335 (6), 377-384.
Swanson et al., Antimicrob. Agents Chemother. 2015, 59 (6), 3042-3051.
Twomey et al., Nature 1957, 179 (4568), 1013-1015.
Tyagi et al., Proceedings of the National Academy of Sciences 2015, 112 (3), 869-874.
Verma et al., Antimicrobial agents and chemotherapy 2013, 57(2): 1050-1052.
Yoon et al., Mol. Pharm. 2015, 12 (7), 2517-2527.
Yoon et al., Antimicrob. Agents Chemother. 2016, 60 (6), 3470-3479.
Zhang et al., Emerging Microbes & Infections 2017, 6 (4), e28-e28.

1. A pharmaceutical composition comprising micronized, crystalline clofazimine particles with a median particle diameter of 0.5 to 10 μm, wherein the composition comprises less than 10% amorphous material, wherein the composition is substantially free of excipients, and wherein the clofazimine is in the non-salt form.

2. (canceled)

3. The pharmaceutical composition of claim 1, wherein the composition is a dry powder.

4-20. (canceled)

21. The pharmaceutical composition of claim 1, wherein the composition comprises 100% by weight of the micronized clofazimine particles.

22. The pharmaceutical composition of claim 1, wherein the micronized clofazimine particles have a median particle diameter of 0.5 to 5 μm.

23-24. (canceled)

25. The pharmaceutical composition of claim 1, wherein at least 80% of the micronized clofazimine particles have a volume equivalent diameter of 1 to 3 μm.

26. The pharmaceutical composition of claim 1, wherein the composition has a specific surface area of 1.9 to 2.3 m²/g.

27. The pharmaceutical composition of claim 1, wherein the composition has a compressibility index of 32 to 37.

28. The pharmaceutical composition of claim 1, wherein the composition has a Hausner ratio of 10 to 20.

29. The pharmaceutical composition of claim 1, wherein the composition has an angle of response of 15° to 30°.

30. (canceled)

31. The pharmaceutical composition of claim 1, wherein the composition comprises a fine particle fraction (FPF) of at least 50%.

32. (canceled)

34. The pharmaceutical composition of claim 1, wherein the composition comprises a dissolution rate of less than 30% in 24 hours in phosphate buffered saline pH 7.4 with 0.2% polysorbate 80 dissolution medium.

35. The pharmaceutical composition of claim 1, wherein the composition comprises less than 5% amorphous material.

36. The pharmaceutical composition of claim 1, wherein the composition is substantially free of amorphous material.

37-38. (canceled)

39. The pharmaceutical composition of claim 1, wherein the composition is produced by jet milling.

40-54. (canceled)

54. A dry powder inhaler comprising a unit dosage form of micronized, crystalline clofazimine particles, wherein the particles comprise a median particle diameter of 0.5 to 10 μm, wherein the clofazimine is in the non-salt form, and the unit dosage form is substantially free of excipients.

55-62. (canceled)

63. The dry powder inhaler of claim 54, wherein the dry powder inhaler comprises an air flow resistance of 0.02 kPa^{0.5} min/L and 0.04 kPa^{0.5} min/L.

64-69. (canceled)

70. A method of preparing the composition of claim 1, comprising:
(b) subjecting clofazimine crystals to a jet mill, wherein the clofazimine is in the non-salt form; and
(c) collecting micronized clofazimine particles with a median particle diameter of 0.5 to 10 μm, wherein the method does not comprise the addition of an excipient.

71-74. (canceled)

75. A method for treating or preventing a pulmonary infection in a patient comprising administering an effective amount of the composition of claim 1 to the patient.

76-110. (canceled)
